(19) United States
(12) Patent Application Publication
(10) Pub. No.: US 2024/0127970 A1
(43) Pub. Date: Apr. 18, 2024

(54) PSYCHOLOGICAL COUNSELING SERVICE METHOD AND USER TERMINAL

(71) Applicant: POCKET HEALING INC., Asan-si Chungcheongnam-do (KR)

(72) Inventor: Hoon KIM, Asan-si Chungcheongnam-do (KR)

(73) Assignee: POCKET HEALING INC., Asan-si Chungcheongnam-do (KR)

(21) Appl. No.: 18/531,923

(22) Filed: Dec. 7, 2023

Related U.S. Application Data
(63) Continuation of application No. PCT/KR2021/017100, filed on Nov. 19, 2021.

(30) Foreign Application Priority Data
Jun. 10, 2021 (KR) 10-2021-0075653
Oct. 15, 2021 (KR) 10-2021-0137701

(51) Int. Cl. G16H 80/00; G16H 20/70

(52) U.S. Cl. CPC G16H 80/00; G16H 20/70

(57) ABSTRACT

Proposed are a psychological counseling service method and a user terminal. The psychological counseling service method includes connecting to the counseling service server to open a group counseling room and outputting a preset basic messenger window for the group counseling room, and dividing the basic messenger window into a plurality of windows according to input of a counseling start command, and transmitting, receiving, and outputting counseling chat messages according to progress of counselling between a counselor and a counselee through the plurality of windows resulting from division, and merging the plurality of windows resulting from division according to input of a counseling termination command and returning to and displaying the basic messenger window.

### CROSS-REFERENCE TO RELATED APPLICATIONS

This application is a continuation of International Application No. PCT/KR2021/017100 filed on Nov. 19, 2021, which claims priority to Korean Patent Application No. 10-2021-0075653 filed on Jun. 10, 2021 and Korean Patent Application No. 10-2021-0137701 filed on Oct. 15, 2021, the entire contents of which are herein incorporated by reference.

### TECHNICAL FIELD

The present disclosure relates to a psychological counseling service method and a user terminal.

### BACKGROUND ART

Online group counselling is efficient in terms of time and cost, because the counselor is in contact with multiple counselees. It is easier and more comfortable to receive online group counselling than individual counselling. Online group counselling enables the group members to freely discuss their issues. In addition, group counselling can provide opportunities to enhance social skills and interpersonal relationships, and can be used for people with lower vocational maturity.

However, since multiple people share personal information in group counselling, it is difficult to guarantee confidentiality. It is also difficult to provide in-depth counselling for each member, and provide appropriate counselling to all members or satisfy them. In addition, for group counselling, forming issue-specific groups can be challenging in terms of time, and there is a possibility of the group's pressure leading to a loss of individuality.

In addition, online group counseling may cause confusion and significantly reduce the members' sensation of immersion as multiple people generate many chat messages simultaneously. In addition, it is difficult for online group counseling to give in-depth counselling for each member and to provide appropriate counselling to all member or satisfy them.

Therefore, there is a need to develop a psychological counseling service method for addressing these problems.

### DISCLOSURE

### Technical Problem

The present disclosure is directed to providing a psychological counseling service method and a user terminal that provide a group counselling service online, wherein anonymity is guaranteed, in-depth interviews are possible through one-on-one counselling, there are no constraints of time, and supplementation is made for each individual management to maximize an individual's individuality.

In addition, the present disclosure is directed to providing a psychological counseling service method and a user terminal that provide a group counseling service online, wherein group counseling is performed using a pop-up window displayed on a basic messenger window, and a plurality of expert groups are connected to group counseling in progress so that group counseling is performed through a plurality of groups.

### Technical Solution

According to an aspect of the present disclosure, there is provided a psychological counseling service method performed by user terminals interworking with a counseling service server.

According to an embodiment, the psychological counseling service method includes: connecting to the counseling service server to open a group counseling room, and outputting a preset basic messenger window for the group counseling room; dividing the basic messenger window into a plurality of windows according to input of a counseling start command; transmitting, receiving, and outputting counseling chat messages according to progress of counselling between a counselor and a counselee through the plurality of windows resulting from division; and merging the plurality of windows resulting from division according to input of a counseling termination command, and returning to the basic messenger window and displaying the basic messenger window.

In the dividing of the basic messenger window into the plurality of windows, the basic messenger window may be divided into a counselling window in an upper position and an observation window in a lower position, and expression icons for observers in the observation window to express their feelings or intentions in real time during the counselling may be displayed in the counselling window, in the observation window, or between the counselling window and the observation window, wherein in the counselling window, a chat may take place between the counselor and the counselee who are set as counselling targets, and in the observation window, a chat may take place between the observers including a plurality of the counselees excluding the counselee set as the counselling target.

In the dividing of the basic messenger window into the plurality of windows, when the user terminals are terminals of the counselor and the counselee in the middle of the counselling, the counselling window and the expression icons may be displayed except the observation window, or when the user terminals are terminals of the observers, the counselling window, the observation window, and the expression icons may be displayed.

In the dividing of the basic messenger window into the plurality of windows, the basic messenger window may be divided into a leader window in an upper position and a member window in a lower position, and expression icons for general members in the member window to express their feelings or intentions in real time during the counselling may be displayed in the leader window, in the member window, or between the leader window and the member window, wherein the counselor may belong to the leader window as a leader, and a plurality of the counselees who are the general members may belong to the member window.

In the dividing of the basic messenger window into the plurality of windows, the basic messenger window may be divided into a main window in an upper position and a sub window in a lower position, wherein the counselor or one counselee designated among a plurality of the counselees who are the general members may belong to the main window as a leader, and the plurality of the counselees who are the general members or a plurality of the counselees excluding the one designated counselee may belong to the sub window.

According to another aspect of the present disclosure, there is provided a user terminal interworking with a counseling service server to provide a psychological counseling service.

According to an embodiment of the present disclosure, the user terminal includes: a memory configured to store instructions therein; and a processor configured to execute the instructions, wherein the instructions include: connecting to the counseling service server to open a group counseling room, and outputting a preset basic messenger window for the group counseling room; dividing the basic messenger window into a plurality of windows according to input of a counseling start command; transmitting, receiving, and outputting counseling chat messages according to progress of counselling between a counselor and a counselee through the plurality of windows resulting from division; and merging the plurality of windows resulting from division according to input of a counseling termination command, and returning to the basic messenger window and displaying the basic messenger window.

According to still another aspect of the present disclosure, there is provided a psychological counseling service method performed by user terminals interworking with a counseling service server.

According to another embodiment of the present disclosure, the psychological counseling service method includes: connecting to the counseling service server to create a self-help group room, and outputting a preset basic messenger window for the self-help group room; creating a pop-up chat room by forming a pop-up chat window on the basic messenger window according to input of a story posting request; transmitting, receiving, and outputting counseling chat messages according to progress of counselling between a counselor and a counselee who has posted a story, through the output pop-up chat window and the basic messenger window; and deleting the pop-up chat room according to input of a counseling termination command, and returning to the basic messenger window and displaying the basic messenger window.

The pop-up chat window may be formed in a preset size at an upper area of the basic messenger window, and may be configured to display the story and the counseling chat messages of the counselee who has posted the story.

In the transmitting, receiving, and outputting of the counseling chat messages according to the progress of the counselling between the counselor and the counselee who has posted the story, the counseling chat messages of the counselee who has posted the story may be output through the pop-up chat window, and the counseling chat messages of the counselor may be output on the basic messenger window according to occurrence of the counseling chat messages of the counselor.

The psychological counseling service method may further include: forming an integrated pop-up chat room by allowing at least one preset expert group to participate in the pop-up chat room according to input of an expert group connection request; and transmitting, receiving, and outputting counseling chat messages according to progress of counselling between the counselee who has posted the story, the counselor, and a plurality of experts, through a pop-up chat window and a temporary pop-up window created in real time output from the formed integrated pop-up chat room.

In the transmitting, receiving, and outputting of the counseling chat messages according to the progress of the counselling between the counselee who has posted the story, the counselor, and the plurality of experts, the pop-up chat window may be created in a preset size at an upper area of a screen, the story and the counseling chat messages of the counselee who has posted the story may be output to the created pop-up chat window, and according to occurrence of the counseling chat messages of the counselor or the experts, the temporary pop-up window displaying the counseling chat messages of the counselor or the experts may be immediately created and output at a lower area under the pop-up chat window.

In the transmitting, receiving, and outputting of the counseling chat messages according to the progress of the counselling between the counselee who has posted the story, the counselor, and the plurality of experts, when the counseling chat messages of the counselor, the counselee, and the experts are output as the expert group participates, a group identifier of the group creating the output counseling chat messages may be displayed and the group inputting the counseling chat messages may be identified in real time.

According to still another aspect of the present disclosure, there is provided a user terminal interworking with a counseling service server to provide a psychological counseling service.

According to another embodiment of the present disclosure, the user terminal includes: a memory configured to store instructions therein; and a processor configured to execute the instructions, wherein the instructions include: connecting to the counseling service server to create a self-help group room, and outputting a preset basic messenger window for the self-help group room; creating a pop-up chat room by forming a pop-up chat window on the basic messenger window according to input of a story posting request; transmitting, receiving, and outputting counseling chat messages according to progress of counselling between a counselor and a counselee who has posted a story, through the output pop-up chat window and the basic messenger window; and deleting the pop-up chat room according to input of a counseling termination command, and returning to the basic messenger window and displaying the basic messenger window.

### Advantageous Effects

The psychological counseling service method and the user terminal according to the embodiments of the present disclosure provide a group counselling service online, wherein anonymity is guaranteed, in-depth interviews are possible through one-on-one counselling, there are no constraints of time, and supplementation is made for each individual management to maximize an individual's individuality.

In addition, the psychological counseling service method and the user terminal according to other embodiments of the present disclosure provide a group counseling service online, wherein group counseling is performed using a pop-up window displayed on a basic messenger window, and a plurality of expert groups are connected to group counseling in progress so that group counseling is performed through a plurality of groups. Accordingly, the psychological counseling service method and the user terminal can provide a high sensation of immersion and diverse user conveniences, and can improve the quality of counselling through professional opinions in various fields.

### DESCRIPTION OF DRAWINGS

FIG. 1 is a diagram schematically illustrating the configuration of a psychological counseling service system according to an embodiment of the present disclosure.

FIG. 2 is a flowchart illustrating a psychological counseling service method according to an embodiment of the present disclosure.

FIGS. 3, 4(a), 4(b), 4(c), 5, and 6 are diagrams illustrating the psychological counseling service method according to the embodiment of the present disclosure shown in FIG. 2.

FIG. 7 is a flowchart illustrating a psychological counseling service method according to another embodiment of the present disclosure.

FIGS. 8 to 11 are diagrams illustrating the psychological counseling service method according to the embodiment of the present disclosure shown in FIG. 7.

FIG. 12 is a diagram schematically illustrating the configuration of a user terminal configured to perform a psychological counseling service method according to an embodiment of the present disclosure.

### MODE FOR INVENTION

In the present specification, an expression used in the singular encompasses the expression of the plural, unless it has a clearly different meaning in the context. In the present specification, the terms “composed of” or “include” should not be construed as necessarily including all of several elements or steps described herein, and accordingly, should be construed as not including some of the several elements or steps or as further including additional elements or steps. In addition, the term “˜part” or “˜module” used in the specification may mean a unit for processing at least one function or operation, and may be realized in hardware or software or may be realized by a combination of hardware and software.

Hereinafter, various embodiments of the present disclosure will be described with reference to the accompanying drawings.

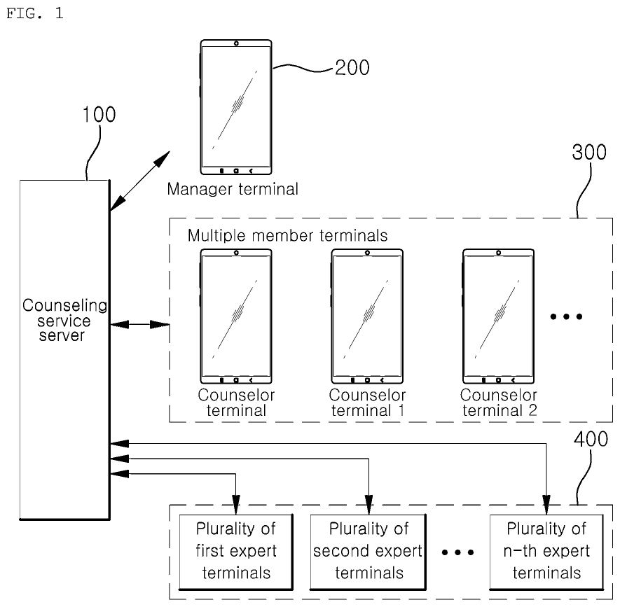

FIG. 1 is a diagram schematically illustrating the configuration of a psychological counseling service system according to an embodiment of the present disclosure.

Referring to FIG. 1, the psychological counseling service system according to the embodiment of the present disclosure may include a counseling service server 100, a manager terminal 200, multiple member terminals 300, and a plurality of expert terminals 400.

The counseling service server 100 is a server that provides a psychological counseling service according to the embodiment of the present disclosure. The server registers and manages a manager, multiple members, and multiple experts, who are users of the manager terminal 200, the multiple member terminal 300, and the plurality of expert terminals 400, and provides the registered manager, members, and experts with various group counseling services.

The manager terminal 200 is the manager's terminal who manages the counseling service server 100 such that the psychological counseling service according to the embodiment of the present disclosure is provided to the registered members.

For example, the counseling service server 100 may configure and open a group counseling room according to the manager's open request and control through the manager terminal 200, and may relay message transmission and reception between the members through the multiple member terminals 300 connecting to the opened group counseling room.

In addition, according to another embodiment of the present disclosure, the counseling service server 100 may open a self-help group room according to the manager's self-help group room creation request and control through the manager terminal 200, and may relay message transmission and reception between the members and the experts through the multiple member terminals 300 and the plurality of expert terminals 400 connecting to the opened self-help group room.

The multiple member terminals 300 may include, as shown in FIG. 1, at least one counselor terminal and a plurality of counselee terminals. Herein, a counselor terminal is a counselor's terminal who gives a counselee counselling, and a counselee terminal is a counselee's terminal who receives counselling from a counselor.

For example, the multiple member terminals 300 are equipped with a messenger application for providing the psychological counseling service according to the embodiment of the present disclosure, and interwork with the counseling service server 100 to provide the counselor and the counselees with the psychological counseling service according to the embodiment of the present disclosure.

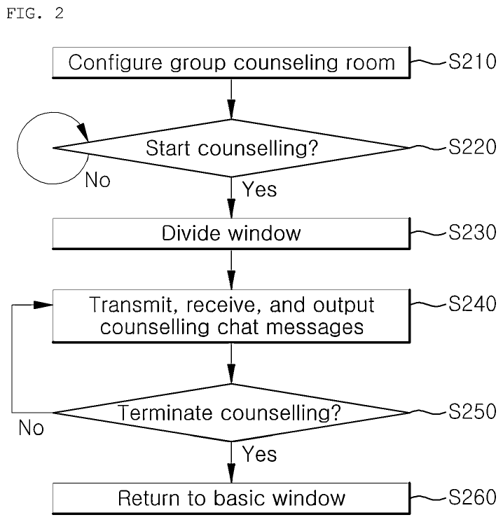

The psychological counseling service according to the embodiment of the present disclosure will be described in detail with reference to FIGS. 2 to 6.

The plurality of expert terminals 400 may include, as shown in FIG. 1, a plurality of first expert terminals, a plurality of second expert terminals, . . . , a plurality of n-th expert terminals. That is, the plurality of expert terminals 400 of field-specific expert groups composed of a plurality of experts for each field may connect to the counseling service server 100.

For example, the multiple member terminals 300 and the plurality of expert terminals 400 are equipped with a messenger application for providing the psychological counseling service according to another embodiment of the present disclosure, and interwork with the counseling service server 100 to provide the counselor, the counselees, and the experts with the psychological counseling service according to the embodiment of the present disclosure.

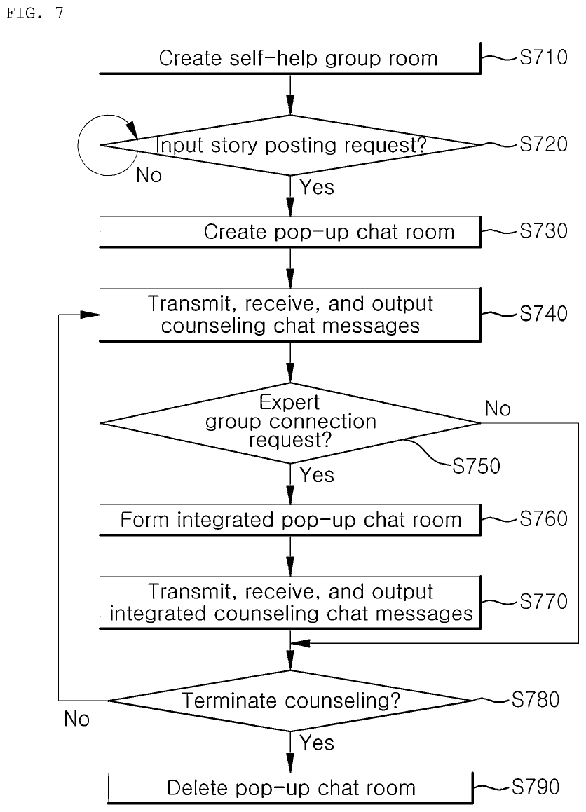

The psychological counseling service according to the embodiment of the present disclosure will be described in detail with reference to FIGS. 7 to 11.

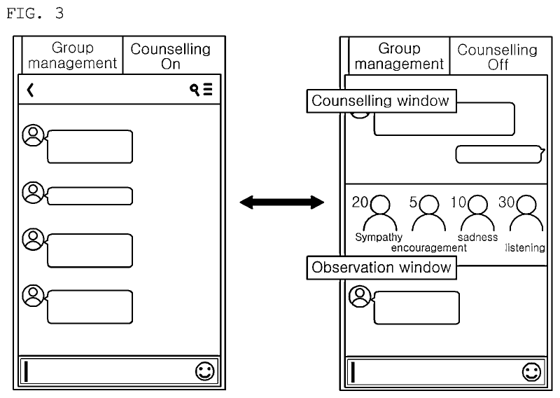

FIG. 2 is a flowchart illustrating a psychological counseling service method according to an embodiment of the present disclosure. FIGS. 3 to 6 are diagrams illustrating the psychological counseling service method according to the embodiment of the present disclosure shown in FIG. 2. Hereinafter, the psychological counseling service method according to the embodiment of the present disclosure will be described focusing on FIG. 2, and FIGS. 3 to 6 will be referenced.

In step S210, the manager terminal 200 connects to the counseling service server 100 to open a group counseling room, and at least one counselor terminal and a plurality of counselee terminals among the multiple member terminals 300 connect to the counseling service server 100 such that the at least one counselor and the plurality of counselees participate in the opened group counseling room. Herein, the manager terminal 200 also participates in the opened group counseling room to configure the counselor and the counselees and manage the group counseling room.

In step S220, the member terminals 300 connected to the counseling service server 100, that is, the at least one counselor terminal and the plurality of counselee terminals, determine whether to start counselling.

For example, FIG. 3 shows an example of a messenger window of the opened group counseling room. As shown on the left of FIG. 3, the manager terminal 200, the counselor terminal, and the counselee terminals output a basic messenger window of the opened group counseling room and display messages written by the manager, the at least one counselor, and the plurality of counselees participating in the group counseling room. Herein, an input window is displayed at the bottom of the basic messenger window, and letters, numbers, symbols, and icons may be input through the input window. In addition to the input window, icons for providing functions, such as a video call and a voice call, may be displayed in the messenger window. The functions may be options to open a one-on-one room for increasing convenience. In addition, the manager terminal 200, the counselor terminal, and the counselee terminals may display a counselling button for inputting a counseling start command or a counseling termination command at the top of the output basic messenger window.

In step S230, the counselor terminal and the counselee terminals divide the previously output basic messenger window into a plurality of windows according to the input of the counseling start command.

For example, when the manager inputs the counseling start command through the counselling button displayed on the manager terminal 200, the manager terminal 200, the counselor terminal, and the counselee terminals may divide the basic messenger window into a counselling window in the upper position and an observation window in the lower position for display as shown on the right of FIG. 3. Herein, in the counselling window, a chat takes place between the counselor and the counselee set as counselling targets. In the observation window, a chat takes place among the observers including the remaining plurality of counselees except the counselee set as the counselling target. In addition, the manager may use the manager terminal 200 to set the preset counselor and counselee to move to the counselling window and set himself or herself to move to the observation window. Herein, the manager terminal 200, the counselor terminal, and the counselee terminals may display expression icons for the observers in the observation window to express their feelings or intentions in real time during counselling so that the counselor and the counselees view the feelings or intentions simultaneously. Herein, the expression icons may include sympathy, encouragement, sadness, and listening icons as shown in FIG. 3.

For example, the expression icons may be displayed between the counselling window and the observation window as shown on the right of FIG. 3. In addition, the expression icons may be displayed on the left, right, top, or right of the counselling window or the observation window, without any limitations on position.

In step S240, the counselor terminal and the counselee terminals transmit and receive and output counseling chat messages according to the progress of counselling between the counselor and the counselee through the plurality of windows resulting from division.

For example, in FIG. 3, only the counselor and the counselee set as the counselling targets are allowed to chat in the counselling window, and at least one counselee may be involved. In addition, real-time chat messages in the observation window are not displayed in the counselling window in order not to disturb counselling between the counselor and the counselee.

The observers in the observation window may leave opinions to the manager through the observation window or one-on-one writing to the manager, and the manager may feed the observers' opinions back to the counselor when counselling ends.

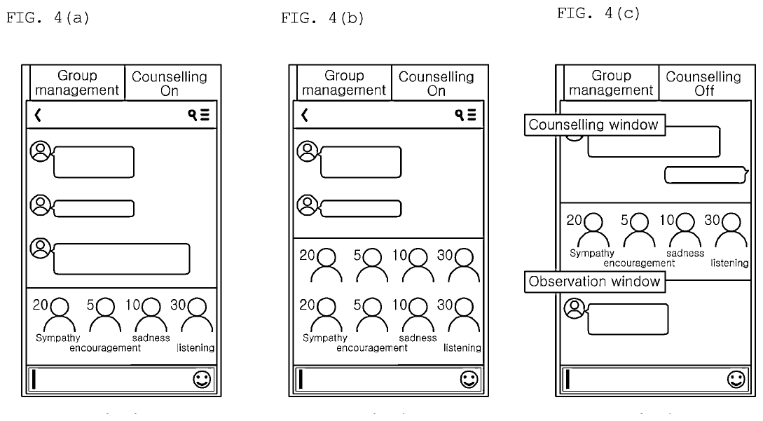

In the meantime, FIGS. 4(a), 4(b), 4(c) shows an example of a messenger window of a group counseling room according to another embodiment of the present disclosure. That is, the observation window does not need to be displayed on the counselor terminal of the counselor and the counselee terminal of the counselee who are in the middle of counselling, so only the counselling window and the expression icons may be displayed as shown in FIGS. 4(a) and 4(b). As shown in FIG. 4(a), only the minimum number of expression icons may be set to be displayed so that the observation window is displayed wide. Alternatively, as shown in FIG. 4(b), a large number of expression icons may be set to be displayed so that many expressions are given. In addition, the terminals of the observers who are observing in the observation window may display all the counselling window, the observation window, and the expression icons as shown in FIG. 4(c).

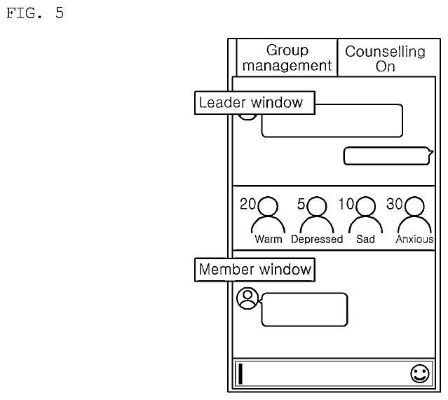

In the meantime, FIG. 5 shows an example of a messenger window of a group counseling room according to still another embodiment of the present disclosure. More specifically, FIG. 5 shows the example of the messenger window of the group counseling room when one-to-many counselling is performed.

Referring to FIG. 5, when the manager inputs a counseling start command for one-to-many counselling through the manager terminal 200, the manager terminal 200, the counselor terminal, and the counselee terminals may divide the basic messenger window into a leader window in the upper position and a member window in the lower position for display as shown in FIG. 5. Herein, the counselor or one counselee designated among the plurality of counselees who are general members may be a leader and may belong to the leader window. In addition, a plurality of counselees who are the remaining general members may belong to the member window. Herein, the manager may freely move the general members belonging to the member window to the leader window or may freely move the counselor or the general member belonging to the leader window to the member window. Through this, the content provided through posts or links written by the leader may not disappear and may be fixed on the full screen and always visible to the general members. In addition, expression icons for the general members of the member window to express their feelings or intentions in real time may be displayed so that the leader and the members view the feelings or intentions simultaneously.

For example, the expression icons may be displayed between the leader window and the member window as shown in FIG. 5. In addition, the expression icons may be displayed on the left, right, top, or right of the leader window or the member window, without any limitations on position.

Through this, the leader may check the states of the members. That is, the counselor may show management statistics of the group of counselees on the psychology of the group of counselees through psychological counseling for the group of counselees, and may receive feedback on the provided counselling article or content. In addition, the counselor may introduce additional missions or information to the group of counselees through the leader window.

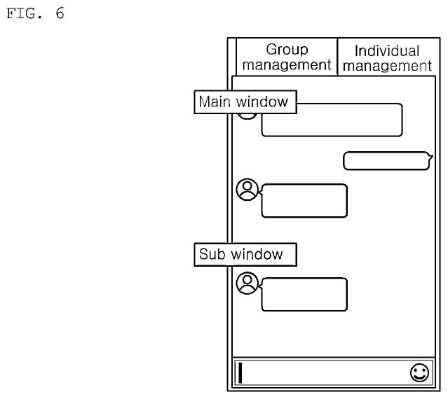

In the meantime, FIG. 6 shows an example of a messenger window of a group counseling room according to still another embodiment of the present disclosure. More specifically, FIG. 6 shows the example of the messenger window of the group counseling room when used for sharing general content rather than psychological counseling.

Referring to FIG. 6, when the basic messenger window is set by the manager to be divided into a main window in the upper position and a sub window in the lower position, the manager terminal 200, the counselor terminal, and the counselee terminals may divide the messenger window into the main window in the upper position and the sub window in the lower position for display as shown in FIG. 6. Herein, the counselor or one counselee designated among the plurality of counselees who are general members may be a leader and may belong to the main window. In addition, the remaining plurality of general members may belong to the sub window. Herein, the manager may freely move the general members belonging to the sub window to the leader window or may freely move the counselor or the general member belonging to the main window to the sub window.

Through this, the leader may collect various opinions regarding analysis data using a group, and may share the analysis data, and may make use as a chat platform for transmitting and receiving data such as video. In addition, the multiple general members may include a group of experts in the same field or various fields, so the group of experts may perform a simple exchange of opinions and real-time voting is possible, enabling quick decision-making.

In step S250, the counselor terminal and the counselee terminals determine whether to terminate counselling.

For example, when counselling between the counselor and the counselee ends, the manager may input the counseling termination command through the counselling button displayed on the manager terminal 200.

In step S260, the counselor terminal and the counselee terminals merge the plurality of windows resulting from division according to the input of the counseling termination command and return to the basic messenger window and display the same.

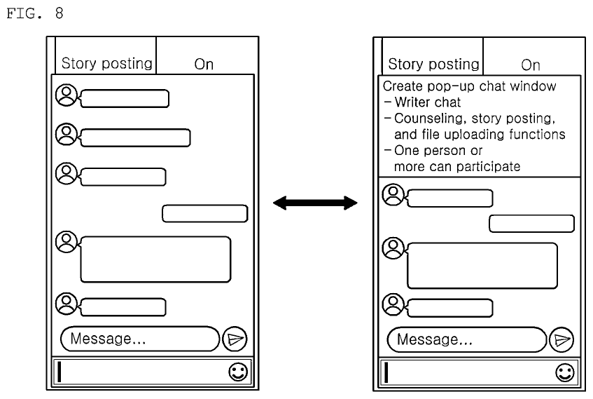

FIG. 7 is a flowchart illustrating a psychological counseling service method according to another embodiment of the present disclosure. FIGS. 8 to 11 are diagrams illustrating the psychological counseling service method according to the embodiment of the present disclosure shown in FIG. 7. Hereinafter, the psychological counseling service method according to the embodiment of the present disclosure will be described focusing on FIG. 7, and FIGS. 8 to 11 will be referenced.

In step S710, the manager terminal 200 connects to the counseling service server 100 to open a self-help group room, and at least one counselor terminal and a plurality of counselee terminals among the multiple member terminals 300 connect to the counseling service server 100 to participate in the opened self-help group room. Herein, the manager terminal 200 also participates in the opened self-help group room to configure the counselor and the counselees and manage the self-help group room.

In step S720, the member terminals 300 connected to the counseling service server 100, that is, the at least one counselor terminal and the plurality of counselee terminals, determine whether to input a story posting request.

For example, FIG. 8 shows an example of a messenger window of the created self-help group room. As shown on the left of FIG. 8, the manager terminal 200, the counselor terminal, and the counselee terminals output a basic messenger window of the opened self-help group room and display messages written by the manager, the at least one counselor, and the plurality of counselees participating in the self-help group room. Herein, an input window is displayed at the bottom of the basic messenger window, and letters, numbers, symbols, and icons may be input through the input window. In addition to the input window, icons for providing functions, such as a video call and a voice call, may be displayed in the messenger window.

In addition, the manager terminal 200, the counselor terminal, and the counselee terminals may display a story posting button for inputting the story posting request at the top of the output basic messenger window.

In step S730, the counselor terminal and the counselee terminals create a pop-up chat room by forming a pop-up chat window on the messenger window of the self-help group room according to the input of the story posting request.

For example, when one of the plurality of counselees inputs a story posting request through the story posting button on the counselee terminal to post a story for counselling, the manager terminal 200, the counselor terminal, and the counselee terminal may create the pop-up chat window in a preset size at an upper area of the messenger window of the self-help group room as shown on the right of FIG. 8. In addition, the story input by the counselee may be displayed in the pop-up chat window and fixed. The pop-up chat window is an exclusive chat window for a counselee who has posted a story. The pop-up chat window may display not only the story but also the counseling chat messages input by the counselee, and may provide functions such as uploading files. Herein, one or more counselees may post stories simultaneously, and accordingly, the one or more counselees who have posted stories may use the pop-up chat room as an exclusive chat window.

In step S740, the counselor terminal and the counselee terminals transmit and receive and output counseling chat messages according to the progress of counselling between the counselor and the counselee who has posted the story, through the output pop-up chat window and the messenger window of the self-help group room.

That is, the counselor terminal and the counselee terminals may transmit and receive counseling chat messages according to the progress of counselling between the counselor and the counselee who has posted the story, and may output counseling chat messages of the counselee who has posted the story through the created pop-up chat window, and may output counseling chat messages of the counselor through the messenger window of the self-help group room when the counseling chat messages of the counselor occur.

Herein, the remaining counselees excluding the counselee who has posted the story among the plurality of counselees may watch counselling between the counselor and the counselee who has posted the story. In addition, at least one of the remaining counselees may input counseling chat messages and participate in counselling as a type of a counselor.

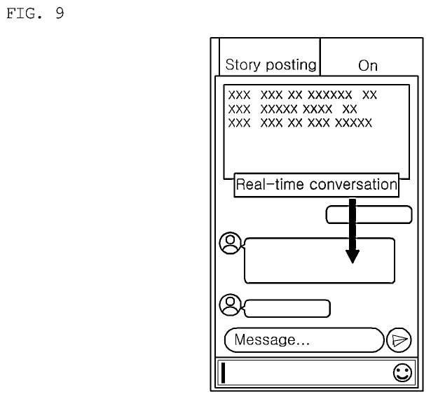

For example, FIG. 9 shows an example of a screen in which real-time conversation takes place in the pop-up chat room. Referring to FIG. 9, the counseling chat messages of the counselee who has posted the story are displayed in the pop-up chat window in the preset size output at the upper area of the messenger window of the self-help group room, and according to the occurrence of counseling chat messages of the counselor or other counselees, the counseling chat messages of the counselor may be displayed in the messenger window of the self-help group room. That is, the messenger window of the self-help group room may be created at a first layer on the screen of the counselor terminal and the counselee terminals, and the pop-up chat window may be created at a second layer on the screen. Herein, the pop-up chat window created at the second layer may be fixed and displayed, and the latest counseling chat messages of the counselor may be displayed in the messenger window of the self-help group room at an area of the first layer not covered by the pop-up chat window at the second layer. Herein, the counselor may be determined among the plurality of counselees, and may be at least one of the plurality of counselees.

In step S750, the counselor terminal and the counselee terminals determine whether to input an expert group connection request.

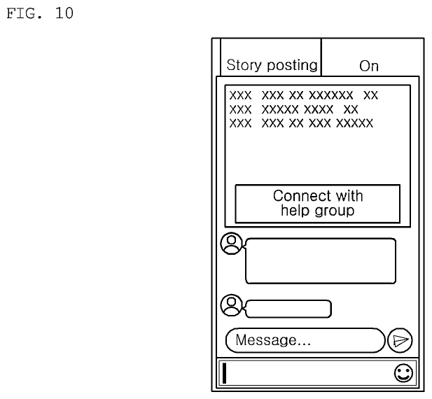

For example, FIG. 10 shows an example of the screen outputting the pop-up chat window in which a group connection button is displayed. Referring to FIG. 10, in order to connect an expert group to the pop-up chat room, the group connection button for requesting connection to the expert group may be formed at the bottom of the pop-up chat window.

In step S760, the counselor terminal and the counselee terminals form an integrated pop-up chat room by allowing at least one preset expert group to participate in the pop-up chat room according to the input of the expert group connection request.

In advance, the plurality of expert terminals 400 of field-specific expert groups composed of a plurality of experts for each field may connect to the counseling service server 100. In addition, a plurality of expert terminals 400 of each expert group may create an expert group room to stand by in the expert group room in order to participate in a group counseling service for multi- and interdisciplinary problem analysis of a self-help group according to an embodiment of the present disclosure.

In step S770, the counselor terminal, the counselee terminals, and the expert terminals transmit and receive and output counseling chat messages according to the progress of counselling between the counselee who has posted the story, the counselor, and the plurality of experts, through a pop-up chat window and a temporary pop-up window created in real time output from the formed integrated pop-up chat room.

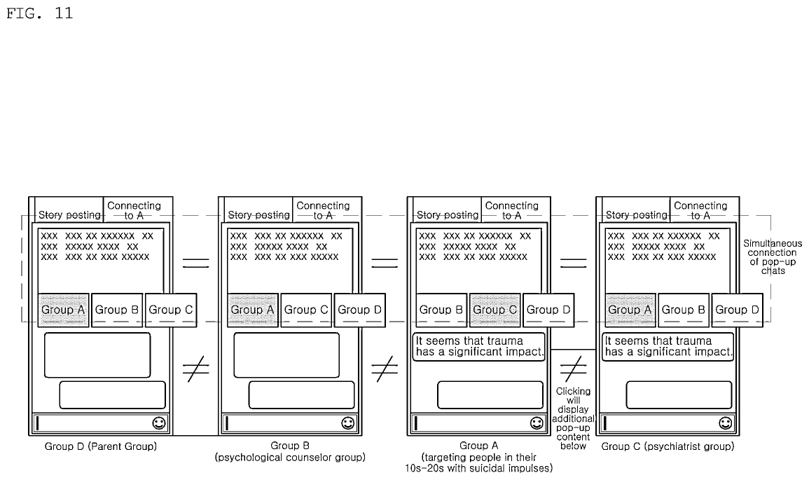

For example, FIG. 11 shows an example of a screen in which real-time conversation is made in the integrated pop-up chat room. In FIG. 11, group A is a group composed of at least one counselor and a plurality of counselees. The screen of the counselor terminal and the counselee terminals in group A shows a real-time conversation between the counselees, the counselor, and the plurality of experts taking place through the integrated pop-up chat room formed as the plurality of expert groups participate, during counselling between the counselor and the counselee who has posted a story about suicidal impulses in people in their teens and twenties through the above-described pop-up chat room. In addition, group B, group C, and group D show examples of the expert groups. That is, group B is a group of psychological counselors, group C is a group of psychiatrists, and group D is a group of parents. The screen of the expert terminals in group B, group C, and group D may display the same real-time conversation between the counselees, the counselor, and the plurality of experts taking place through the integrated pop-up chat room as the screen of the counselor terminal and the counselee terminals in group A.

However, in the integrated pop-up chat room, when the counseling chat messages of the counselor, the counselee, and the experts are output through the pop-up chat window or the temporary pop-up window as the expert groups participate, a group identifier of the group creating the output counseling chat messages is displayed and the group inputting the counseling chat messages may be identified in real time.

That is, referring to FIG. 11, the counselor terminal, the counselee terminals, and the expert terminals in group A, group B, group C, and group D create the pop-up chat window, like the pop-up chat room, in the preset size at the upper area of the screen and output a story and counseling chat messages of the counselee who has posted the story to the pop-up chat window. According to the occurrence of counseling chat messages of the counselor or the experts, the temporary pop-up window displaying the counseling chat messages of the counselor or the experts may be immediately created and output at a lower area under the pop-up chat window. Herein, as shown in FIG. 11, a plurality of pop-up icons of groups other than the user may be displayed between the pop-up chat window and the temporary pop-up window. In addition, among the plurality of pop-up icons, the pop-up icons of the group creating the output counseling chat messages may be displayed in an emphasized manner. The plurality of pop-up icons may be used when a group is designated and counseling chat messages are transmitted only to the designated group. That is, among the plurality of pop-up icons, when the pop-up icon of the group to which counseling chat messages are to be transmitted is touched, the pop-up icon is displayed in an emphasized manner and counseling chat messages input afterward may be transmitted to the terminals in the group of which the pop-up icon is touched.

In step S780, the counselor terminal, the counselee terminals, or the expert terminals determine whether to terminate counselling.

For example, when counselling between the counselor and the counselee ends or counselling between the counselor, the counselee, and the experts ends, the manager may input the counseling termination command through the counselling button on the manager terminal 200.

In step S790, the counselor terminal, the counselee terminals, or the expert terminals delete the pop-up chat room or the integrated pop-up chat room according to the input of the counseling termination command and return to the basic messenger window of the self-help group room and display the same.

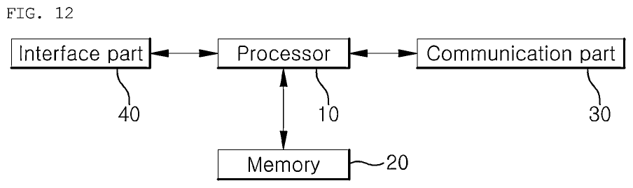

FIG. 12 is a diagram schematically illustrating the configuration of a user terminal configured to perform a psychological counseling service method according to an embodiment of the present disclosure.

Referring to FIG. 12, the user terminal according to the embodiment of the present disclosure includes a processor 10, a memory 20, a communication part 30, and an interface part 40.

The processor 10 may be a CPU or a semiconductor device that executes processing instructions stored in the memory 20.

The memory 20 may include various types of volatile or non-volatile storage media. For example, the memory 20 may include ROM or RAM.

For example, the memory 20 may store instructions for performing the psychological counseling service method according to the embodiment of the present disclosure.

The communication part 30 is a means for transmitting and receiving data to and from other devices over a communication network.

The interface part 40 may include a network interface for connecting to the network and a user interface.

In the meantime, the elements of the above-described embodiments can be easily understood from the perspective of processes. That is, each of the elements may be understood as a separate process. In addition, the processes of the above-described embodiments can be easily understood from the perspective of the elements of an apparatus.

In addition, the above-described technical content may be realized in the form of program instructions executable by various computer means and may be recorded on a computer-readable recording medium. The computer-readable recording medium may include program instructions, data files, data structures, and the like separately or in combinations. The program instructions to be recorded on the computer-readable recording medium may be specially designed and configured for the embodiments of the present disclosure or may be well-known to and be usable by those skilled in the art of computer software. Examples of the computer-readable recording medium include magnetic recording media such as hard disks, floppy disks and magnetic tapes; optical data storage media such as CD-ROMs or DVD-ROMs; magneto-optical media such as floptical disks; and hardware devices, such as read-only memory (ROM), random-access memory (RAM), and flash memory, which are particularly structured to store and implement the program instruction. Examples of the program instructions include not only a mechanical language code formatted by a compiler but also a high-level language code that may be implemented by a computer using an interpreter, and the like. The hardware devices may be configured to be operated by one or more software modules or vice versa to conduct the operation according to the embodiments.

The above-embodiments of the present disclosure have been described for illustrative purposes, and those skilled in the art will be able to make various modifications, changes, and additions within the spirit and scope of the present disclosure, and such modifications, changes, and additions should be regarded as falling within the scope of the claims below.

1. A psychological counseling service method performed by user terminals interworking with a counseling service server, the psychological counseling service method comprising:
connecting to the counseling service server to open a group counseling room, and outputting a preset basic messenger window for the group counseling room;
dividing the basic messenger window into a plurality of windows according to input of a counseling start command;
transmitting, receiving, and outputting counseling chat messages according to progress of counselling between a counselor and a counselee through the plurality of windows resulting from division; and
merging the plurality of windows resulting from division according to input of a counseling termination command, and returning to the basic messenger window and displaying the basic messenger window.

2. The psychological counseling service method of claim 1, wherein in the dividing of the basic messenger window into the plurality of windows,
the basic messenger window is divided into a counselling window in an upper position and an observation window in a lower position, and
expression icons for observers in the observation window to express their feelings or intentions in real time during the counselling are displayed in the counselling window, in the observation window, or between the counselling window and the observation window,
wherein in the counselling window, a chat takes place between the counselor and the counselee who are set as counselling targets, and
in the observation window, a chat takes place between the observers including a plurality of the counselees excluding the counselee set as the counselling target.

3. The psychological counseling service method of claim 2, wherein in the dividing of the basic messenger window into the plurality of windows,
when the user terminals are terminals of the counselor and the counselee in the middle of the counselling, the counselling window and the expression icons are displayed except the observation window, or
when the user terminals are terminals of the observers, the counselling window, the observation window, and the expression icons are displayed.

4. A group counseling service method performed by user terminals interworking with a counseling service server, the group counseling service method being a psychological counseling service method and comprising:
connecting to the counseling service server to create a self-help group room, and outputting a preset basic messenger window for the self-help group room;
creating a pop-up chat room by forming a pop-up chat window on the basic messenger window according to input of a story posting request;
transmitting, receiving, and outputting counseling chat messages according to progress of counselling between a counselor and a counselee who has posted a story, through the pop-up chat window and the basic messenger window; and
deleting the pop-up chat room according to input of a counseling termination command, and returning to the basic messenger window and displaying the basic messenger window.

5. The group counseling service method of claim 4, wherein the pop-up chat window
is formed in a preset size at an upper area of the basic messenger window, and
is configured to display the story and the counseling chat messages of the counselee who has posted the story.

6. The group counseling service method of claim 4, wherein in the transmitting, receiving, and outputting of the counseling chat messages according to the progress of the counselling between the counselor and the counselee who has posted the story,
the counseling chat messages of the counselee who has posted the story are output through the pop-up chat window, and
the counseling chat messages of the counselor are output on the basic messenger window according to occurrence of the counseling chat messages of the counselor.

7. A user terminal interworking with a counseling service server to provide a psychological counseling service, the user terminal performing a psychological counseling service method and comprising:
a memory configured to store instructions therein; and
a processor configured to execute the instructions,
wherein the instructions comprise:
connecting to the counseling service server to open a group counseling room, and outputting a preset basic messenger window for the group counseling room;
dividing the basic messenger window into a plurality of windows according to input of a counseling start command;
transmitting, receiving, and outputting counseling chat messages according to progress of counselling between a counselor and a counselee through the plurality of windows resulting from division; and
merging the plurality of windows resulting from division according to input of a counseling termination command, and returning to the basic messenger window and displaying the basic messenger window.

8. A user terminal interworking with a counseling service server to provide a psychological counseling service, the user terminal performing a psychological counseling service method and comprising:
a memory configured to store instructions therein; and
a processor configured to execute the instructions,
wherein the instructions comprise:
connecting to the counseling service server to create a self-help group room, and outputting a preset basic messenger window for the self-help group room;
creating a pop-up chat room by forming a pop-up chat window on the basic messenger window according to input of a story posting request;
transmitting, receiving, and outputting counseling chat messages according to progress of counselling between a counselor and a counselee who has posted a story, through the output pop-up chat window and the basic messenger window; and
deleting the pop-up chat room according to input of a counseling termination command, and returning to the basic messenger window and displaying the basic messenger window.
